(19) United States
(12) Patent Application Publication
(10) Pub. No.: US 2023/0365760 A1
(43) Pub. Date: Nov. 16, 2023

(54) METHOD FOR PREPARING MULTIFUNCTIONAL HYDROGEL BY YEAST FERMENTATION

(71) Applicant: FUZHOU UNIVERSITY, Fuzhou (CN)

(72) Inventors: Shaoyun WANG, Fuzhou (CN); Jing CHENG, Fuzhou (CN); Lijun YOU, Fuzhou (CN); Caihua XIONG, Fuzhou (CN); Qianqian CAI, Fuzhou (CN); Xuan CHEN, Fuzhou (CN)

(73) Assignee: FUZHOU UNIVERSITY, Fuzhou (CN)

(21) Appl. No.: 18/034,062

(22) PCT Filed: Aug. 18, 2022
(86) PCT No.: PCT/CN2022/113331

§ 371 Date: Apr. 27, 2023

(30) Foreign Application Priority Data
Sep. 6, 2021 (CN) 202111038363.0

(51) Int. Cl. C08J 3/075; C08K 3/04; A61B 5/259; A61L 26/00; A61K 9/00

(52) U.S. Cl. CPC C08J 3/075; C08K 3/042; A61B 5/259; A61L 26/0023; A61L 26/008; A61K 9/0009; A61L 26/0038; C08J 2305/12; C08J 2489/00

(57) ABSTRACT

A method for preparing a multifunctional hydrogel by yeast fermentation is provided. According to the present disclosure, graphene oxide is reduced by polydopamine to obtain a reduced graphene oxide solution first, and then a certain concentration of a gelatin-PCA-glucose mixed solution and an activated yeast liquid are prepared. The three solutions are uniformly mixed and stirred by a one pot reaction method, poured into a mold, and subjected to fermentation in a 30° C. water bath for a certain period of time, so as to obtain a Gel-PrGO-PCA-yeast multifunctional hydrogel material. The method is simple, convenient, rapid, and efficient. The obtained hydrogel has good air permeability, superior mechanical properties, electrical conductivity, and biocompatibility, and can be applied to different skin locations to achieve electrocardiograph and electromyographic detection.

### CROSS REFERENCE TO THE RELATED APPLICATIONS

This application is the national phase entry of International Application No. PCT/CN2022/113331, filed on Aug. 18, 2022, which is based upon and claims priority to Chinese Patent Application No. 202111038363.0, filed on Sep. 6, 2021, the entire contents of which are incorporated herein by reference.

### TECHNICAL FIELD

The present disclosure belongs to the field of preparation of porous conductive hydrogels, and specifically relates to a method for preparing a multifunctional hydrogel by yeast fermentation.

### BACKGROUND

As a material with a three-dimensional polymer and supramolecular polymer network structure, hydrogel has good flexibility, and can be pulled, pressed, and bent. Conductive hydrogels are prepared by adding conductive polymer for physical crosslinking. Due to unique properties (such as flexibility, high water content, biocompatibility, and electrical conductivity), hydrogel has been widely used in a variety of biomedical fields, including detection of human physical signals, regenerative medicine, and nerve repair. However, the conductive hydrogels have poor air permeability, mechanical properties, and water-retaining property, which is greatly limited their application in the field of biomedical. Therefore, it is great significance to improve the air permeability, mechanical properties, and water-retaining property of hydrogel.

Biosensors are important device for detecting and tracking physiological signals of the human body, and hydrogel is a novel biosensor. The hydrogel has high comfortableness, shape controllability, and sensitivity for detecting and tracking electrocardiograph, electromyographic, electroencephalogram and nerve signals of the human body. Most of current hydrogels have impermeability and poor mechanical strength. Therefore, preparation of a porous air-permeable conductive hydrogel with high strength has a broad development and application prospect. Moreover, preparation of currently reported conductive hydrogels includes biologically incompatible synthetic polymers, a toxic crosslinking agent, and complex operation processes. Therefore, development of a simple, rapid, safe, and efficient method to prepare air-permeable hydrogel has great significance.

### SUMMARY

In view of the research defects in the above field, an objective of the present disclosure is to provide a method for preparing a multifunctional hydrogel by yeast fermentation. The method has simple, rapid, and efficient operation. The obtained hydrogel has good air permeability, water-retaining property, flexibility, and electrical conductivity.

In order to achieve the above objective, the following technical schemes are adopted:

A method for preparing a multifunctional hydrogel by yeast fermentation includes the following steps:
(1) activating yeast in a 30° C. warm water to obtain a yeast liquid;
(2) weighing plate count agar (PCA) and dissolving in deionized water at 100° C. for 20 minutes, then cooling to 50° C. to obtain a PCA solution; adding gelatin (Gel) to the PCA solution at stirring in a 50° C. bath for 30 minutes; and then adding glucose, conducting stirring continuously for dissolution to obtain a Gel-PCA-glucose mixed solution, and lowering the temperature to 30° C.;
(3) subjecting the yeast solution in step (1) and the Gel-PCA-glucose mixed solution in step (2) to uniform mixing to obtain a mixture, pouring the mixture into a mold and putting in a 30° C. water bath for fermentation 30 minutes, and then placing at 4° C. for 10 minutes to obtain Gel-PCA-yeast multifunctional hydrogel.

In step (1), the yeast liquid has a concentration of 0.2 g/mL to 0.45 g/mL.

In step (2), the PCA solution has a concentration of 0.0235 g/mL; the Gel is added in an amount of 5 wt % to 35 wt %; and the glucose is added in an amount of 0.01-0.06 g/mL.

Further, in step (2), reduced graphene oxide (PrGO) with a concentration of 1-4 mg/mL is added to the PCA solution to obtain a Gel-PrGO-PCA-glucose mixed solution. The yeast liquid in step (1) and the Gel-PrGO-PCA-glucose mixed solution are subjected to uniform mixing and stirring to obtain a mixture, and the mixture is poured into a mold for fermentation at 30° C. water bath for 30 minutes and then placed at 4° C. for 10 minutes to obtain a Gel-PrGO-PCA-yeast multifunctional hydrogel.

Furthermore, a preparation method of the PrGO includes:
(1) preparation of graphene oxide (GO): weighing 1.2 g of graphite, adding 50 mL of concentrated sulfuric acid, and conducting uniform stirring to obtain a mixture; putting the mixture in an ice bath, adding 1.5 g of sodium nitrate under stirring, slowly adding 6 g of potassium permanganate, conducting stirring at 35° C. overnight, and slowly adding 100 mL of deionized water for a reaction at 90° C. for 1 hour; slowly adding 30 vol % of hydrogen peroxide that is diluted 5 times to the above solution until no bubbles are produced, cooling to room temperature after continuous reaction for 3 hours, washing with water to neutral, and then ultrasonic dispersion for 20 minutes and freeze drying to obtain a GO powder;
(2) dissolving 20 mg GO powder in deionized water, and conducting ultrasonic treatment until complete dissolution to obtain GO dispersion; dissolving 50 mg dopamine hydrochloride (DA) powder in 2.5 mL of 10 mM Tris-HCl solution (pH=8.5) to obtain a DA dispersion; and then adding the DA dispersion to the GO dispersion, conducting ultrasonic dispersion in an ice bath for 2 hours, and then conducting stirring in a 60° C. water bath for 12 hours to obtain PrGO solution.

Further, in step (3), the obtained Gel-PCA-yeast hydrogel is frozen at −80° C. for 20 minutes, cut into a slice with a thickness of 1 mm or cut in an arbitrary shape, and soaked in a mixed solution of a saline solution and glycerol for 12 hours. The saline solution is an ammonium sulfate or sodium citrate solution; the saline solution has a concentration of 10 wt % to 30 wt %; and a volume ratio of the saline solution to the glycerol is 2:1, 1:1, or 1:2.

A multifunctional hydrogel is prepared by any one of the above methods.

The multifunctional hydrogel is used as a conductive material in biosensors, or in drug loadings and antibacterial wound dressings.

Compared with the prior art, the present disclosure has the following advantages:
(1) In the present disclosure, the Gel, the RGO, the PCA, the yeast, and the glucose are mixed for preparation of porous hydrogel by fermentation, and then an obtained hydrogel is soaked in a saline solution or a mixed solution of a saline solution and glycerol.
(2) The multifunctional hydrogel prepared by yeast fermentation has a variety of functions. Due to the yeast, the hydrogel is endowed with porous air permeability. Due to the Gel, the PrGO, and the saline solution, the hydrogel is endowed with electrical conductivity and mechanical properties.
(3) The hydrogel has fatigue resistance and a superior tensile property, and the tensile property can reach 1000%. Moreover, the hydrogel can be used for sensitively detecting electrocardiograph and electromyographic signals, and is expected to be applied in biosensors, wearable devices or other fields.

### BRIEF DESCRIPTION OF THE DRAWINGS

FIG. 1 shows the influence on pore size when different contents of Gel is added.

FIGS. 2A-2D are diagrams showing the morphology of hydrogels. FIG. 2A is a diagram showing the morphology of a hydrogel without addition of yeast under an ordinary camera; FIG. 2B is a diagram showing the morphology of the hydrogel without addition of yeast under an optical microscope; FIG. 2C is a diagram showing the morphology of a hydrogel with addition of yeast under an ordinary camera; and FIG. 2D is a diagram showing the morphology of the hydrogel with addition of yeast under an optical microscope.

FIG. 3 shows tensile stress-strain curves of a Gel-PCA-yeast-ammonium sulfate hydrogel and a Gel-PCA-yeast-sodium citrate hydrogel soaked in different concentrations of salt solutions of ammonium sulfate or sodium citrate.

FIG. 4 is a Young's modulus diagram.

FIGS. 5A-5D are detection diagrams showing the morphology, electrical conductivity, and electrocardiograph and electromyographic signals of a Gel-PrGO-PCA-yeast hydrogel. FIG. 5A shows the morphology of the Gel-PrGO-PCA-yeast hydrogel; FIG. 5B shows the detection of the electrical conductivity; FIG. 5C shows the detection of the electrocardiograph signals; and FIG. 5D shows the detection of the electromyographic signals.

FIG. 6 shows the water-retaining property of a Gel-PCA-yeast hydrogel and Gel-PCA-yeast-ammonium sulfate-glycerol hydrogels.

FIG. 7 shows the rheological property of a Gel-PCA-yeast hydrogel and a Gel-PCA-yeast-ammonium sulfate-glycerol hydrogel.

### DETAILED DESCRIPTION OF THE EMBODIMENTS

The present disclosure is further explained in combination with the embodiments below. It should be pointed out that the embodiments are merely used to explain the present disclosure, rather than to limit the present disclosure.

### Example 1

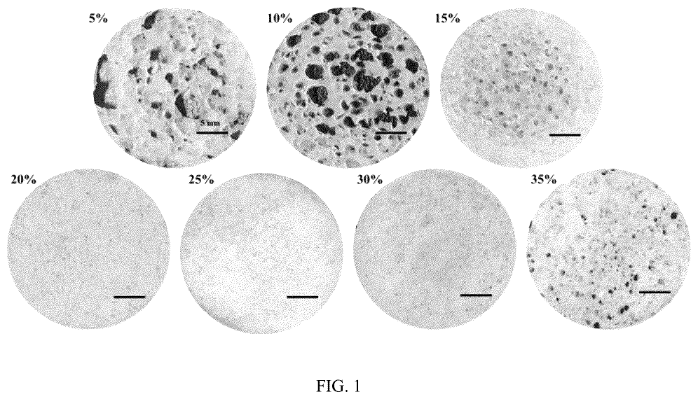

7 portions of 0.235 g PCA were separately dissolved in 9 mL of deionized water at 100° C. for 20 minutes, and cooled to 50° C. 5 wt %, 10 wt %, 15 wt %, 20 wt %, 25 wt %, 30 wt %, and 35 wt % of Gel were separately added, and stirred for 30 minutes in a 50° C. water bath. Then, 0.15 g of glucose was added, and stirred continuously for dissolution to obtain a Gel-PCA-glucose mixed solution. The temperature was lowered to 30° C. 0.45 g yeast powder was dissolved in 1 mL of deionized water at 30° C. Next, the obtained yeast solution and the Gel-PCA-glucose mixed solution were subjected to uniform mixing by stirring to obtain a mixture. The mixture was poured into a mold for fermentation in a 30° C. water bath for 30 minutes, and then placed at 4° C. for 10 minutes to obtain a Gel-PCA-yeast hydrogel. At last, the hydrogel was frozen at −80° C. for 20 minutes, cut into a slice with a thickness of 1 mm or cut in an arbitrary shape, and soaked in 20 wt % of a mixed solution of ammonium sulfate and glycerol (at a volume ratio of 1:1) for 12 hours for detection. As shown in FIG. 1, with the increase of the content of the Gel, the pore size of the Gel-PCA-yeast hydrogel obtained is first decreased and then increased.

### Example 2

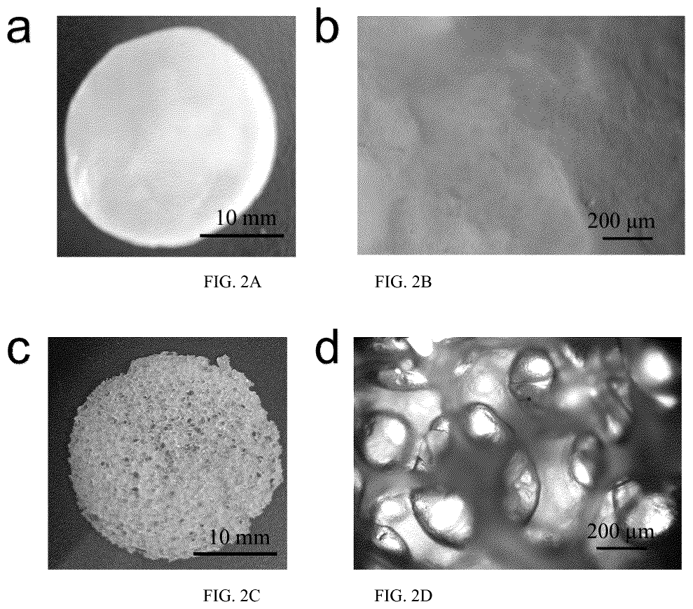

0.235 g of PCA was dissolved in 9 mL of deionized water at 100° C. for 20 minutes and cooled to 50° C. 20 wt % of Gel was added and stirred in a 50° C. water bath for 30 minutes. Then, 0.15 g of glucose was added and stirred continuously for dissolution to obtain a Gel-PCA-glucose mixed solution. The temperature was lowered to 30° C. 0.45 g of a yeast powder was dissolved in 1 mL of deionized water at 30° C. (yeast was not added in a control group). Next, an obtained yeast solution and the Gel-PCA-glucose mixed solution were subjected to uniform mixing and stirring to obtain a mixture. The mixture was poured into a mold for fermentation in a 30° C. water bath for 30 minutes, and then placed at 4° C. for 10 minutes to obtain a Gel-PCA-yeast hydrogel. At last, the hydrogel was frozen at −80° C. for 20 minutes and cut into a slice with a thickness of 1 mm or an arbitrary shape for detection. As shown in FIGS. 2A-2D, the Gel-PCA-yeast hydrogel obtained has a good porous network structure.

### Example 3

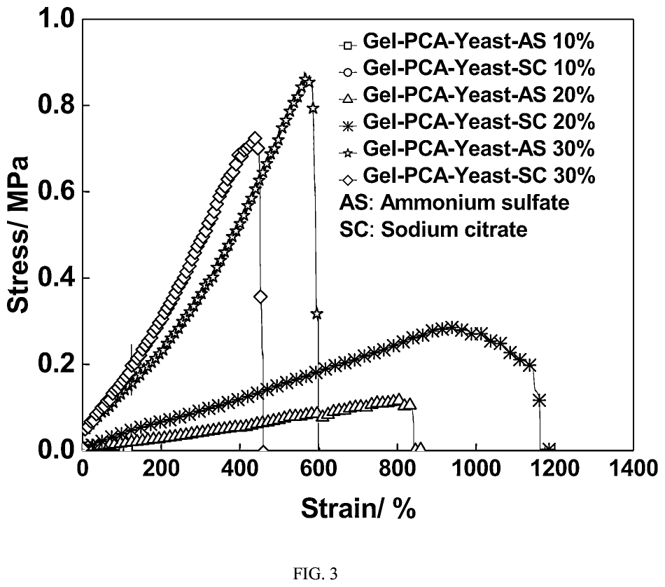

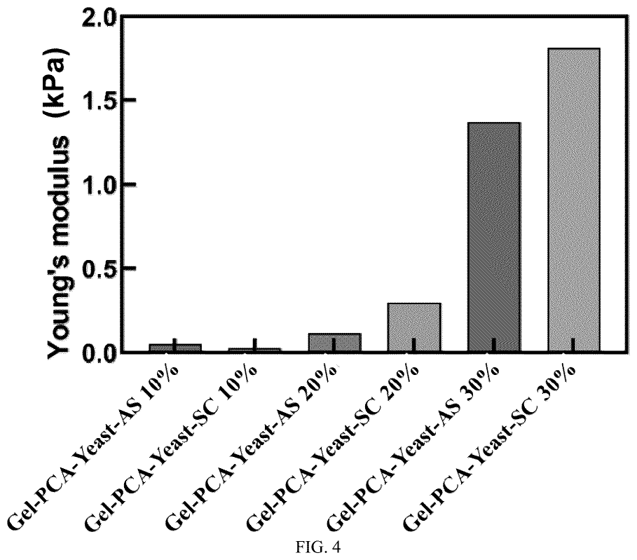

0.235 g of PCA was dissolved in 9 mL of deionized water at 100° C. for 20 minutes and cooled to 50° C. 20 wt % of Gel was added and stirred in a 50° C. water bath for 30 minutes. Then, 0.15 g of glucose was added and stirred continuously for dissolution to obtain a Gel-PCA-glucose mixed solution. The temperature was lowered to 40° C. 0.45 g of a yeast powder was dissolved in 1 mL of deionized water at 30° C. (yeast was not added in a control group). Next, an obtained yeast solution and the Gel-PCA-glucose mixed solution were subjected to uniform mixing and stirring to obtain a mixture. The mixture was poured into a mold for fermentation in a 30° C. water bath for 30 minutes, and then placed at 4° C. for 10 minutes to obtain a Gel-PCA-yeast hydrogel. At last, the hydrogel was frozen at −80° C. for 20 minutes and cut into a slice with a thickness of 1 mm. After being cut into a desired shape, the hydrogel was separately soaked in 10 wt %, 20 wt %, and 30 wt % of salt solutions of ammonium sulfate or sodium citrate for 12 hours, and then the tensile property was tested. As shown in FIG. 3, in a 20% saline solution soaking group, the obtained Gel-PCA-yeast-ammonium sulfate hydrogel and a Gel-PCA-yeast-sodium citrate hydrogel have a superior tensile property. The Gel-PCA-yeast-sodium citrate hydrogel has a maximum tensile strain of 1000% and a maximum tensile stress of 0.28 MPa. The Gel-PCA-yeast-ammonium sulfate hydrogel has a maximum tensile strain of 850% and a maximum tensile stress of 0.14 MPa. Although the tensile property of the sodium citrate group is greater than that of the ammonium sulfate group, the Young's modulus of the ammonium sulfate group is smaller than that of the sodium citrate group (FIG. 4), indicating that the flexibility or elasticity is better. In a 10% saline solution soaking group, hydrogels have poor mechanical properties, and the tensile property is less than 100%.

### Example 4

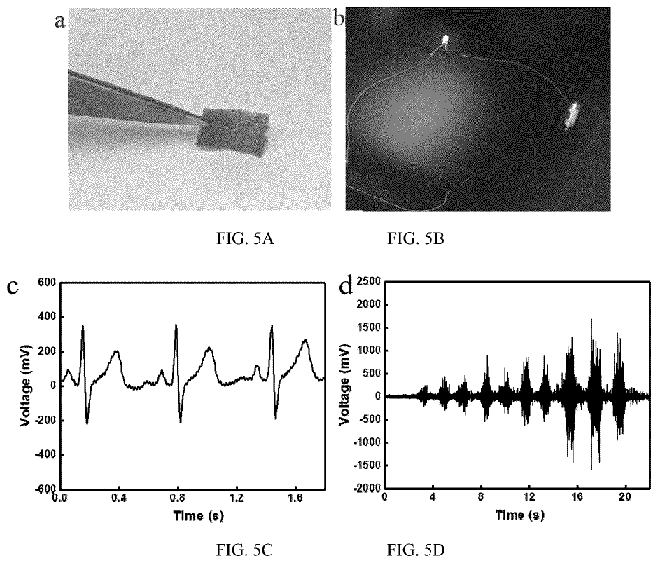

A method for preparing a multifunctional hydrogel by fermentation of yeast includes the following steps:
(1) preparation of graphene oxide (GO): weighing 1.2 g of graphite, adding 50 mL of concentrated sulfuric acid, and conducting uniform stirring to obtain a mixture; putting the mixture in an ice bath, adding 1.5 g of sodium nitrate under stirring, slowly adding 6 g of potassium permanganate, conducting stirring at 35° C. overnight, and slowly adding 100 mL of deionized water for a reaction at 90° C. for 1 hour; slowly adding 30 vol % of hydrogen peroxide that is diluted 5 times to the above solution until no bubbles are produced, cooling to room temperature after continuous reaction for 3 hours, washing with water to neutral, and then ultrasonic dispersion for 20 minutes and freeze drying to obtain a GO powder;
(2) dissolving 20 mg GO powder in deionized water, and conducting ultrasonic treatment until complete dissolution to obtain GO dispersion; dissolving 50 mg dopamine hydrochloride (DA) powder in 2.5 mL of 10 mM Tris-HCl solution (pH=8.5) to obtain a DA dispersion; and then adding the DA dispersion to the GO dispersion, conducting ultrasonic dispersion in an ice bath for 2 hours, and then conducting stirring in a 60° C. water bath for 12 hours to obtain PrGO solution.
(3) dissolving 0.235 g of PCA in 4 mL of deionized water at 100° C., and then cooling to 50° C.; uniformly mixing the above solution with the PrGO solution prepared in step (2), adding 20 wt % of Gel, and conducting stirring in a 50° C. water bath for 30 minutes; then adding 0.15 g of glucose, conducting stirring continuously for dissolution to obtain a Gel-PrGO-PCA-glucose mixed solution, and lowering the temperature to 30° C.; dissolving 0.45 g of a yeast powder in 1 mL of deionized water at 30° C. to obtain a yeast solution; and subjecting the yeast solution and the Gel-PrGO-PCA-glucose mixed solution to uniform mixing to obtain a mixture, pouring the mixture into a mold for fermentation in a 30° C. water bath for 30 minutes, and then placing the mixture at 4° C. for 10 minutes to obtain a Gel-PrGO-PCA-yeast hydrogel. At last, the hydrogel was frozen at −80° C. for 20 minutes. After being cut into a desired shape, the hydrogel was soaked in 20 wt % of ammonium sulfate for 12 hours for next detection. As shown in FIG. 5, the obtained Gel-PrGO-PCA-yeast hydrogel has good electrical conductivity. The hydrogel has a conductivity of 0.015 S/m and a good porous network structure, and can be used for well detecting electrocardiograph and electromyographic signals.

### Example 5

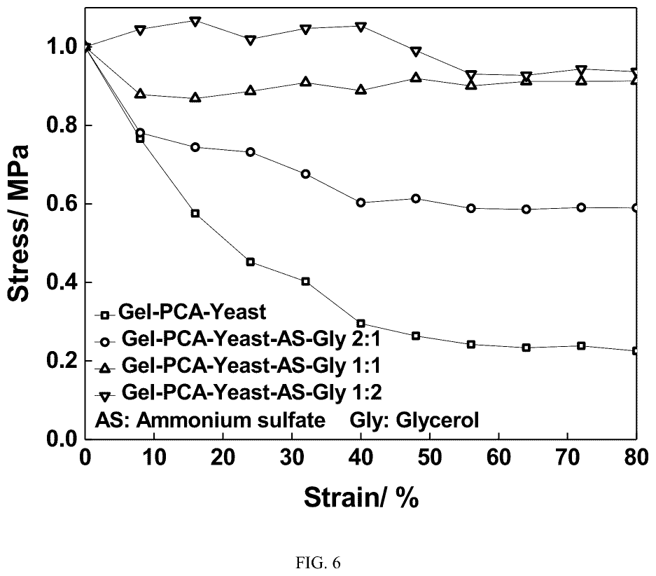

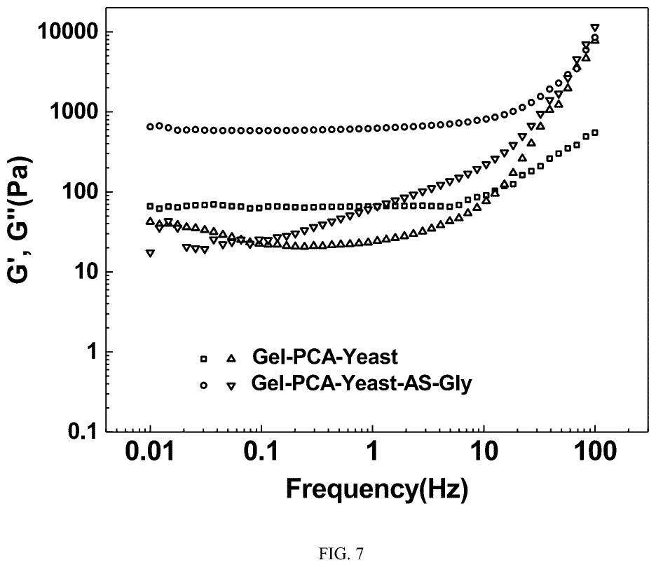

0.235 g of PCA was dissolved in 9 mL of deionized water at 100° C. for 20 minutes and cooled to 50° C. 20 wt % of Gel was added and stirred in a 50° C. water bath for 30 minutes. Then, 0.15 g of glucose was added and stirred continuously for dissolution to obtain a Gel-PCA-glucose mixed solution. The temperature was lowered to 30° C. 0.45 g of a yeast powder was dissolved in 1 mL of deionized water at 30° C. Next, an obtained yeast solution and the Gel-PCA-glucose mixed solution were subjected to uniform mixing and stirring to obtain a mixture. The mixture was poured into a mold for fermentation in a 30° C. water bath for 30 minutes, and then placed at 4° C. for 10 minutes to obtain a Gel-PCA-yeast hydrogel. At last, the hydrogel was frozen at −80° C. for 20 minutes and cut into a slice with a thickness of 1 mm. After being cut into a desired shape, the hydrogel was soaked in 20 wt % of a mixed solution of ammonium sulfate and glycerol (1:2, 1:1, or 2:1 (v/v)) for 12 hours for detection. As shown in FIG. 6 and FIG. 7, the obtained Gel-PCA-yeast-ammonium sulfate-glycerol hydrogel has good water-retaining property and rheological property. When the soaking ratio of the ammonium sulfate to the glycerol is 1:1 and 1:2, the mass is almost unchanged after the hydrogel is placed at room temperature for three days, indicating that less water is lost. According to rheological detection results, it is shown that when the soaking ratio of the ammonium sulfate to the glycerol is 1:1, the energy storage modulus is increased from 100 Pa to 1,000 Pa, which is 1 orders of magnitude higher than that of a non-soaked group, indicating that the soaked hydrogel has higher strength than the non-soaked group.

The foregoing descriptions are merely preferred embodiments of the present disclosure, and all equivalent changes and modifications made according to the scope of the present disclosure for patent application shall fall within the scope of the present disclosure.

What is claimed is:

1. A method for preparing a multifunctional hydrogel by yeast fermentation, comprising the following steps:
(1) activating yeast in 30° C. warm water to obtain a yeast liquid;
(2) weighing plate count agar (PCA) and dissolving in deionized water at 100° C. for 20 minutes, then cooling to 50° C. to obtain a PCA solution; adding gelatin (Gel) to the PCA solution with stirring in a 50° C. bath for 30 minutes; and then adding glucose, conducting the stirring continuously for dissolution to obtain a Gel-PCA-glucose mixed solution, and lowering the temperature of the Gel-PCA-glucose mixed solution to 30° C.;
(3) subjecting the yeast liquid in step (1) and the Gel-PCA-glucose mixed solution in step (2) to uniform mixing to obtain a first mixture, pouring the first mixture into a first mold for fermentation in a 30° C. water bath for 30 minutes, and then placing at 4° C. for 10 minutes to obtain a Gel-PCA-yeast multifunctional hydrogel.

2. The method for preparing the multifunctional hydrogel by yeast fermentation according to claim 1, wherein in step (1), the yeast liquid has a concentration of 0.2 g/mL to 0.45 g/mL.

3. The method for preparing the multifunctional hydrogel by yeast fermentation according to claim 1, wherein in step (2), the PCA in the PCA solution has a concentration of 0.0235 g/mL; the Gel is added in an amount of 5 wt % to 35 wt %; and the glucose is added in an amount of 0.01-0.06 g/mL.

4. The method for preparing the multifunctional hydrogel by yeast fermentation according to claim 1, wherein in step (2), reduced graphene oxide (PrGO) with a concentration of 1-4 mg/mL is added to the PCA solution to obtain a Gel-PrGO-PCA-glucose mixed solution; and the yeast solution in step (1) and the Gel-PrGO-PCA-glucose mixed solution are subjected to uniform mixing and stirring to obtain a second mixture, and the second mixture is poured into a second mold for fermentation in a 30° C. water bath for 30 minutes and then placed at 4° C. for 10 minutes to obtain a Gel-PrGO-PCA-yeast multifunctional hydrogel.

5. The method for preparing the multifunctional hydrogel by yeast fermentation according to claim 4, wherein a preparation method of a PrGO solution comprises the following steps:
(1) preparing graphene oxide (GO) by weighing 1.2 g of graphite, adding 50 mL of concentrated sulfuric acid, and uniformly stirring to obtain a third mixture; putting the third mixture in an ice bath, adding 1.5 g of sodium nitrate under stirring, slowly adding 6 g of potassium permanganate, stirring at 35° C. overnight, and slowly adding 100 mL of the deionized water for a reaction at 90° C. for 1 hour to obtain a solution; slowly adding 30 vol % of hydrogen peroxide, wherein the 30 vol % of hydrogen peroxide is diluted 5 times to the solution until no bubbles are produced, cooling to room temperature after a continuous reaction for 3 hours, washing with water to neutral, and then subjecting to an ultrasonic dispersion for 20 minutes and freeze drying to obtain a GO powder;
(2) dissolving 20 mg GO powder in the deionized water, and conducting an ultrasonic treatment until completely dissolved to obtain a GO dispersion; dissolving 50 mg dopamine hydrochloride (DA) powder in 2.5 mL of 10 mM Tris-HCl solution (pH=8.5) to obtain a DA dispersion; and then adding the DA dispersion to the GO dispersion, conducting an ultrasonic dispersion in an ice bath for 2 hours, and then stirring in a 60° C. water bath for 12 hours to obtain the PrGO solution.

6. The method for preparing the multifunctional hydrogel by yeast fermentation according to claim 1, wherein in step (3), the Gel-PCA-yeast multifunctional hydrogel obtained is frozen at −80° C. for 20 minutes, and then cut into a slice with a thickness of 1 mm or cut in an arbitrary shape, and soaked in a mixed solution of a saline solution and glycerol for 12 hours.

7. The method for preparing the multifunctional hydrogel by yeast fermentation according to claim 6, wherein the saline solution is an ammonium sulfate or sodium citrate solution; the saline solution has a concentration of 10 wt % to 30 wt %; and a volume ratio of the saline solution to the glycerol is 2:1, 1:1, or 1:2.

8. A multifunctional hydrogel prepared by the method according to claim 1.

9. A method of using the multifunctional hydrogel according to claim 8, wherein the multifunctional hydrogel is configured as a conductive material in biosensors, or in drug loadings and antibacterial wound dressings.

10. The multifunctional hydrogel according to claim 8, wherein in step (1), the yeast liquid has a concentration of 0.2 g/mL to 0.45 g/mL.

11. The multifunctional hydrogel according to claim 8, wherein in step (2), the PCA in the PCA solution has a concentration of 0.0235 g/mL; the Gel is added in an amount of 5 wt % to 35 wt %; and the glucose is added in an amount of 0.01-0.06 g/mL.

12. The multifunctional hydrogel according to claim 8, wherein in step (2), a reduced graphene oxide (PrGO) with a concentration of 1-4 mg/mL is added to the PCA solution to obtain a Gel-PrGO-PCA-glucose mixed solution; and the yeast solution in step (1) and the Gel-PrGO-PCA-glucose mixed solution are subjected to uniform mixing and stirring to obtain a second mixture, and the second mixture is poured into a second mold for fermentation in a 30° C. water bath for 30 minutes and then placed at 4° C. for 10 minutes to obtain a Gel-PrGO-PCA-yeast multifunctional hydrogel.

13. The multifunctional hydrogel according to claim 12, wherein a preparation method of a PrGO solution comprises the following steps:
(1) preparing graphene oxide (GO) by weighing 1.2 g of graphite, adding 50 mL of concentrated sulfuric acid, and uniformly stirring to obtain a third mixture; putting the third mixture in an ice bath, adding 1.5 g of sodium nitrate under stirring, slowly adding 6 g of potassium permanganate, stirring at 35° C. overnight, and slowly adding 100 mL of the deionized water for a reaction at 90° C. for 1 hour to obtain a solution; slowly adding 30 vol % of hydrogen peroxide, wherein the 30 vol % of hydrogen peroxide is diluted 5 times to the solution until no bubbles are produced, cooling to room temperature after a continuous reaction for 3 hours, washing with water to neutral, and then subjecting to an ultrasonic dispersion for 20 minutes and freeze drying to obtain a GO powder;
(2) dissolving 20 mg GO powder in the deionized water, and conducting an ultrasonic treatment until completely dissolved to obtain a GO dispersion; dissolving 50 mg dopamine hydrochloride (DA) powder in 2.5 mL of 10 mM Tris-HCl solution (pH=8.5) to obtain a DA dispersion; and then adding the DA dispersion to the GO dispersion, conducting an ultrasonic dispersion in an ice bath for 2 hours, and then stirring in a 60° C. water bath for 12 hours to obtain the PrGO solution.

14. The multifunctional hydrogel according to claim 8, wherein in step (3), the Gel-PCA-yeast multifunctional hydrogel obtained is frozen at −80° C. for 20 minutes, and then cut into a slice with a thickness of 1 mm or cut in an arbitrary shape, and soaked in a mixed solution of a saline solution and glycerol for 12 hours.

15. The multifunctional hydrogel according to claim 14, wherein the saline solution is an ammonium sulfate or sodium citrate solution; the saline solution has a concentration of 10 wt % to 30 wt %; and a volume ratio of the saline solution to the glycerol is 2:1, 1:1, or 1:2.
